(19) United States
(12) Patent Application Publication
(10) Pub. No.: US 2018/0116268 A1
(43) Pub. Date: May 3, 2018

(54) SYNBIOTIC COMPRISING MIRACLE FRUIT AND PROBIOTICS, TABLET THEREOF, AND METHOD FOR PREPARING THE SAME

(71) Applicant: SEN YUH FARM SCIENCE CO., LTD., KAOHSIUNG CITY (TW)

(72) Inventor: Mei-Ling Lin, KAOHSIUNG CITY (TW)

(21) Appl. No.: 15/793,917

(22) Filed: Oct. 25, 2017

(30) Foreign Application Priority Data
Oct. 27, 2016 (TW) 105134665

(51) Int. Cl. A23L 33/135; A23L 2/04; A61K 9/20

(52) U.S. Cl. CPC A23L 33/135; A61K 2035/115; A61K 9/20; A23L 2/04

(57) ABSTRACT

Provided is a method for preparation of a synbiotic having miracle fruit and probiotics, including the following steps: (1) preparing miracle fruit and mixing the miracle fruit with water to obtain a miracle fruit solution; (2) adjusting a pH value of the miracle fruit solution to range from 5 to 8; (3) adding milk powder to the miracle fruit solution after adjusting the pH value to obtain a miracle fruit matrix; and (4) culturing the miracle fruit matrix and the probiotics to obtain the synbiotic having the miracle fruit and the probiotics. Also provided is the synbiotic having miracle fruit and probiotics prepared by the abovementioned method. Also provided is a tablet of synbiotic having miracle fruit and probiotics comprising synbiotic powder, a first fruit powder, and an excipient. The synbiotic having miracle fruit and probiotics can significantly increase SOD-like activity for anti-oxidation.

### CROSS REFERENCE

This application claims priority to Taiwan Patent Application Serial No. 105134665, filed on Oct. 27, 2016, the content of which is hereby incorporated by reference in its entirety.

### BACKGROUND OF THE INVENTION

### 1. Field of the Invention

The present invention relates to a synbiotic, particularly to a synbiotic comprising miracle fruit and probiotics. The present invention also relates to a preparation method, particularly to a method for preparation of a synbiotic comprising miracle fruit and probiotics. The present invention also relates to a tablet, particularly to a tablet of synbiotic comprising miracle fruit and probiotics.

### 2. Description of the Prior Arts

According to the statistics from Taiwan Ministry of Health and Welfare, colorectal cancer has ranked the third of top ten cancers for six years, which is closely related to the modern people's dietary habits. The reasons of the increasing rate of colorectal cancer include westernization of diets, preference of grilled red meat, lack of exercise, and obesity. Besides regularly taking health checkups and increasing exercise, creating a healthy dietary habit is also a way to reduce the risk of cancer. Scientific researches indicate that the human gastrointestinal bacteria will be changed by the host's dietary habits, thereby affecting the host's metabolic function and increasing the incidence of disease. Domestic and international medical studies have confirmed that probiotics and prebiotics have a positive effect on intestinal health. Aging, metabolic syndrome, inflammation, and so forth will affect and decrease people's immunity. The intestine is not only the digestive organ, but also the largest immune organ. Antioxidants can prevent free radicals within the body and reduce the formation of inflammation. Therefore, ant-oxidation has become one of the key factors for delaying aging and reducing inflammation.

Miracle fruit (Synsepalm dulcificum) belonging to Sapotaceae Family is an evergreen shrub, and is also commonly known as “incredible berry”. The miracle fruit originates from the jungle marshes of West African countries such as Congo, Ghana, etc. The indigenous people use the miracle fruit with fermented food which is nauseated, and the miracle fruit can transfer the sour taste of the fruit into sweet taste because of miraculin inside the miracle fruit. Therefore, the miracle fruit is of great value, and many scientists extract the special glycoprotein from the miracle fruit for use in the control of diabetics' diet. The ripe miracle fruit is about 1 cm in diameter and about 2 cm in length, the peel is bright red, and a brown seed is inside the white embryo. The main ingredients of the white embryo are glycoprotein, citric acid, malic acid, succinic acid, etc. The miraculin of the miracle fruit is one of the taste-modifying proteins, and the miraculin can alter the sour taste of the fruit into sweet taste on the taste buds. Although the miracle fruit has the taste-modifying effect, the activity of miraculin will be destroyed below pH 2.5 or above pH 12.0, or in an organic solvent, so that the miracle fruit is hard to be manufactured into tablets for portability.

Therefore, a tablet comprising the miracle fruit that has an anti-oxidation function and is portable for users at the same time is needed to resolve the disadvantage of the prior art should.

### SUMMARY OF THE INVENTION

According to the above description, the objective of the present invention is to provide a synbiotic comprising miracle fruit and probiotics to improve functions of removing superoxide and protecting cells from oxidative damage.

To achieve the above objective of the present invention, the present invention provides a method for preparation of a synbiotic comprising miracle fruit and probiotics, the method comprising the following steps: (1) preparing miracle fruit and mixing the miracle fruit with water to obtain a miracle fruit solution; (2) adjusting a pH value of the miracle fruit solution to range from 5 to 8; (3) adding milk powder to the miracle fruit solution after adjusting the pH value to obtain a miracle fruit matrix; and (4) culturing the miracle fruit matrix and the probiotics to obtain the synbiotic comprising the miracle fruit and the probiotics.

The “synbiotic” refers to that the mixture of probiotics and prebiotics is cultured for a time, and the synbiotic in the gastrointestinal tract can enrich the probiotics, thereby improving health. In the present invention, the synbiotic includes the combination of lactic acid bacteria and the miracle fruit matrix, or the combination of lactic acid bacteria and the miracle fruit matrix, wherein the lactic acid bacteria are selected from the group consisting of Lactobacillus casei, Lactobacillus paracasei subsp., Lactobacillus acidophilus, Streptococcus salivarius subsp. thermophilus, and any combination thereof.

Preferably, the weight percent concentration of the miracle fruit in the miracle fruit solution is from 0.1% to 10% in the step (1).

More preferably, the weight percent concentration of the miracle fruit in the miracle fruit solution is from 0.5% to 5% in the step (1).

Preferably, the pH value of the miracle fruit solution is from 6 to 7 in the step (2).

More preferably, the pH value of the miracle fruit solution is 6.5 in the step (2).

Preferably, the weight percent concentration of the milk powder in the miracle fruit matrix is from 1% to 10% in the step (3).

More preferably, the weight percent concentration of the milk powder in the miracle fruit matrix is 6.5% in the step (3).

Preferably, the time for culture is from 24 to 72 hours in the step (4).

More preferably, the time for culture is 48 hours in the step (4).

Preferably, the method in the step (4) further comprises: processing sterilization after culturing the miracle fruit matrix and the probiotics.

More preferably, the sterilization condition is 121° C. for 15 minutes.

Preferably, the probiotics are selected from the group consisting of Lactobacillus casei, Lactobacillus paracasei subsp., Lactobacillus acidophilus, Streptococcus salivarius subsp. thermophilus, and any combination thereof.

More preferably, Lactobacillus casei includes, but is not limited to, BCRC 17487 (Bioresource Collection and Research Center in Taiwan).

More preferably, Lactobacillus paracasei subsp. includes, but is not limited to, BCRC14023, BCRC17001, BCRC17483, BCRC17488, or any combination thereof.

More preferably, Lactobacillus acidophilus includes, but is not limited to, BCRC16092, BCRC16099, or any combination thereof.

More preferably, Streptococcus salivarius subsp. thermophilus includes, but is not limited to, BCRC13869, BCRC14085, BCRC14086, or any combination thereof.

In another aspect, the present invention relates to a synbiotic comprising miracle fruit and probiotics prepared from a method as above mentioned.

In another aspect, the present invention relates to a tablet of the synbiotic comprising miracle fruit and probiotics, the tablet comprising a synbiotic powder obtained by drying and grinding the synbiotic comprising miracle fruit and probiotics as above mentioned, a first fruit powder, and an excipient.

In accordance with the tablet of the present invention, the excipient and the synbiotic comprising miracle fruit and probiotics are prepared to a suitable tablet. The “excipient” includes, but is not limited to, plasticizer, filler, lubricant, diluent, binder, disintegrant, solvent, surfactant, preservative, sweetener, and viscosity agents; preferably, the plasticizer includes, but is not limited to, corn flour; preferably, the lubricant includes, but is not limited to, silicon dioxide (SiO₂) or resin.

Preferably, the weight percentage of the synbiotic powder in total weight of the tablet is from 0.1% to 1% (w/w).

More preferably, the first fruit powder is miracle fruit powder or miracle fruit-lemon juice powder; wherein the miracle fruit-lemon juice powder is obtained from mixing miracle fruit and lemon juice at a weight ratio ranging from 1:1.5 to 1.5:1 after drying and grinding.

More preferably, the tablet further comprises a second fruit powder, and the second fruit powder is selected from the group consisting of tomato powder, pineapple powder, banana powder, pitaya powder, and any combination thereof.

More preferably, the tablet further includes, but is not limited to, Bacillus, Bifidobacterium, Enterococcus, Lactobacillus, Saccharomyces, Sporolactobacillus, Streptococcus, Clostridium, Monascus, Propionibacterium, Candida, or any combination thereof.

The advantage of the present invention is that the synbiotic comprising miracle fruit and probiotics can significantly increase the superoxide dismutase-like (SOD-like) activity, thereby promoting the removal of superoxide and protecting cells from oxidative damage. Besides, the miracle fruit comprising miraculin can alter the sour taste into sweet taste for developing the tablet of synbiotic comprising miracle fruit and probiotics, thereby obtaining an anti-oxidative, healthy, low-calorie, and sugar-free functional oral tablet.

Other objectives, advantages and novel features of the invention will become more apparent from the following detailed description when taken in conjunction with the accompanying drawings.

### BRIEF DESCRIPTION OF THE DRAWINGS

FIG. 1 is a histogram of total bacteria concentration of ten strains of lactic acid bacteria cultured by de Man, Rogosa and Sharpe (MRS) medium of the present invention; sample codes are shown in Table 1 below.

FIG. 2 is a photograph of miracle fruit matrices of different concentrations after sanitization of the present invention.

FIG. 3 is a histogram of total bacteria concentration of single strain of lactic acid bacteria with the miracle fruit matrix after fermentation of the present invention; sample codes are shown in Table 1 below.

FIG. 4 is a histogram of total bacteria concentration of single strain or mixed strains of lactic acid bacteria with the miracle fruit matrix after fermentation of the present invention; sample codes are shown in Table 1 below.

FIG. 5 is a histogram of SOD-like activity of single strain of lactic acid bacteria with the miracle fruit matrix after fermentation of the present invention; sample codes are shown in Table 1 below.

FIG. 6 is a histogram of SOD-like activity of single strain or mixed strains of lactic acid bacteria with the miracle fruit matrix before or after fermentation of the present invention; sample codes are shown in Table 1 below.

FIG. 7 is a photograph of miracle fruit matrices of different concentrations with Lactobacillus casei LC1 after sanitization of the present invention.

FIG. 8 is a histogram of total bacteria concentration of Lactobacillus casei LC1 of the miracle fruit matrices of different concentrations after fermentation of the present invention.

FIG. 9 is a histogram of SOD-like activity of Lactobacillus casei LC1 with the miracle fruit matrices of different concentrations before or after fermentation of the present invention.

### DETAILED DESCRIPTION OF THE PREFERRED EMBODIMENTS

### Preparation 1 Culturing Strains

Literatures show that lactic acid bacteria are good for the human health, such as improving the absorption of lactose, reducing the occurrence of diarrhea, inhibiting bacteria, lowering cholesterol, anti-cancer and enhancing immunity. Ten lactic acid bacteria obtained from BCRC of Food Industry Research and Development Institute in Taiwan are widely applied in food processing, as shown in Table 1.

TABLE 1
10 strains of lactic acid bacteria

| sample code | strain | Accession No. |
|---|---|---|
| LC1 | Lactobacillus | BCRC17487 |
| | casei | |
| PC2 | Lactobacillus | BCRC14023 |
| PC3 | paracasei subsp. | BCRC17001 |
| PC4 | | BCRC17483 |
| PC5 | | BCRC17488 |
| LA1 | Lactobacillus | BCRC16092 |
| LA2 | acidophilus | BCRC16099 |
| SS1 | Streptococcus | BCRC13869 |
| SS2 | salivarius subsp. | BCRC14085 |
| SS3 | thermophilus | BCRC14086 |

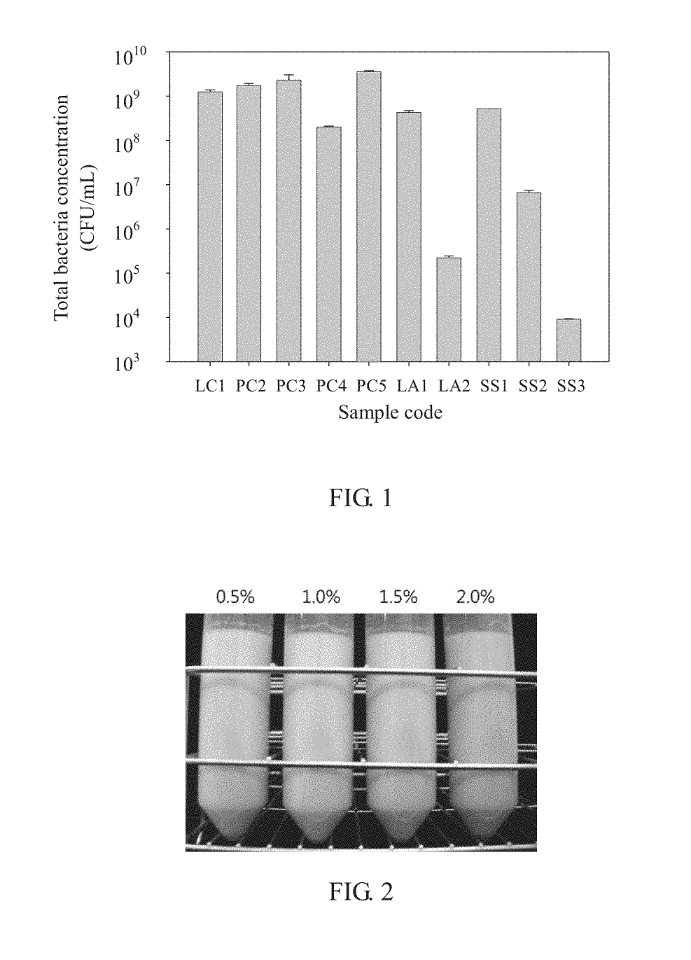

10 mL of each one of the ten lactic acid bacteria having 10⁴ to 10⁹ colony-forming units (CFU/mL) was added and cultured in 100 mL MRS medium at 37° C. for 48 hours. The number of lactic acid bacteria was counted by serial dilution (as shown in FIG. 1). The bacteria numbers of Lactobacillus acidophilus LA2, Streptococcus salivarius subsp. thermophilus SS2 and Streptococcus salivarius subsp. thermophilus SS3 are less than 10⁷ CFU/mL, and the bacteria numbers of the other seven strains are more than 10⁸ CFU/mL.

### Preparation 2 Miracle Fruit Matrix Preparation

Pulp of the miracle fruit with the seed removed was freeze-dried, and then the pulp was ground into miracle fruit powder. The miracle fruit powder was added into water to obtain 0.5%, 1.0%, 1.5%, and 2.0% (w/v) miracle fruit solutions, and the pH values of the miracle fruit solutions were 3.18, 3.21, 3.26, and 3.33, respectively, all below pH 3.5. The miracle fruit solution was neutralized through sodium hydroxide until the pH value was 5 to 8; preferably, pH 6 to 7; more preferably, pH 6.5. 6.5% (w/v) milk powder (28.8 grams of fat per 100 grams of milk powder, purchased from Fernleaf®) added into the miracle fruit solution after neutralization was sterilized at 121° C. for 15 minutes to obtain a miracle fruit matrix (as shown in FIG. 2). If the pH value was not adjusted before sterilization, the miracle fruit matrix will produce a protein denaturation suspension and affect the fermentation procedure of lactic acid bacteria.

### Example 1 Fermentation of Miracle Fruit and Lactic Acid Bacteria

One strain of Lactobacillus casei (LC1), three strains of Lactobacillus paracasei subsp. (PC2, PC3 and PC4), one strain of Lactobacillus acidophilus (LA1), and one strain of Streptococcus salivarius subsp. thermophilus (SS1) were selected from Preparation 1, and 10 mL of concentration 10⁸ CFU/mL to 10⁹ CFU/mL (equaled to 10⁹ CFU to 10¹⁰ CFU) of each strain was cultured with 100 mL of the miracle fruit matrix obtained from Preparation 2 at 37° C. for 48 hours.

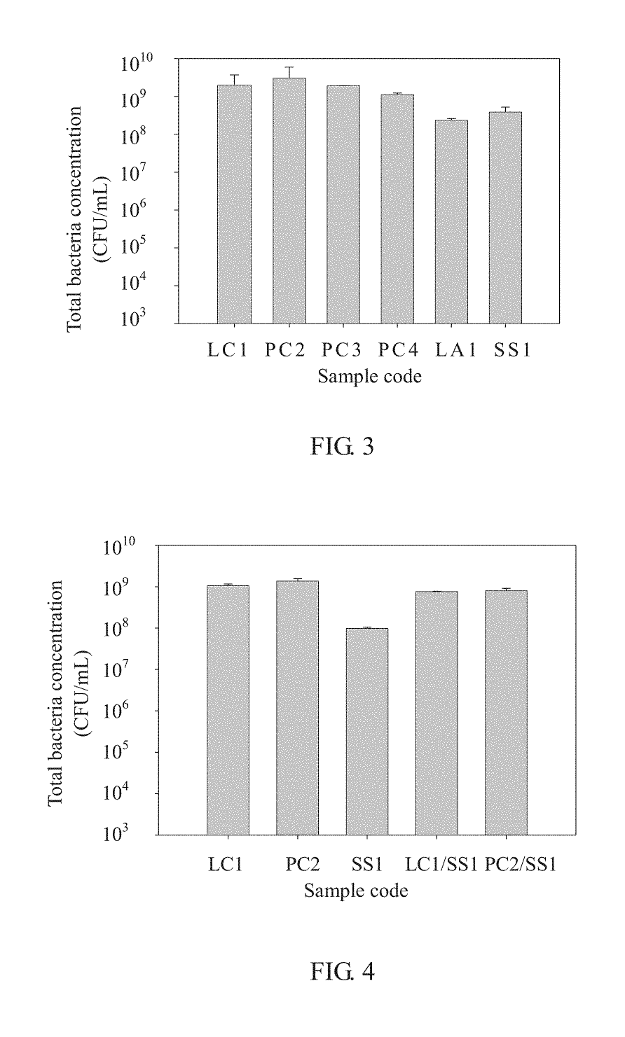

As shown in FIG. 3, total bacteria concentration of each strain is above 10⁸ CFU/mL, the three with the higher bacteria concentration are Lactobacillus paracasei subsp. PC2 3.1×10⁹ CFU/mL, Lactobacillus paracasei subsp. PC3 1.9×10⁹ CFU/mL and Lactobacillus casei LC1 2.0×10⁹ CFU/mL, and the two with the lower bacteria concentration are Lactobacillus acidophilus LA1 2.3×10⁸ CFU/mL and Streptococcus salivarius subsp. thermophilus SS1 3.9×10⁸ CFU/mL.

Besides, five groups including 3 single strain groups and 2 mixed strain groups, Lactobacillus casei LC1, Lactobacillus paracasei subsp PC2, Streptococcus salivarius subsp. Thermophilus SS1, mixed LC1 and SS1, and mixed PC2 and SS1 were selected from Preparation 1 for comparison. The amount of 10⁹ CFU to 10¹⁰ CFU of the single strain group was cultured in 100 mL of 0.5% (w/v) miracle fruit matrix from Preparation 2; LC1 and SS1 of the mixed strain group obtained from 6 mL of 10⁹ CFU/mL LC1 and 4 mL of 10⁸ CFU/mL SS1 was cultured in 100 mL of 0.5% (w/v) miracle fruit matrix from Preparation 2, and the volume ratio of LC1 and SS1 is 3:2 (v/v); PC2 and SS1 of the mixed strain group obtained from 6 mL of 10⁹ CFU/mL PC2 and 4 mL of 10⁸ CFU/mL SS1 was cultured in 100 mL of 0.5% (w/v) miracle fruit matrix from Preparation 2, and the volume ratio of PC2 and SS1 is 3:2 (v/v). The above five groups were cultured at 37° C. for 48 hours.

As shown in FIG. 4, the concentration of LC1 is 1.1×10⁹ CFU/mL, the concentration of PC2 is 1.4×10⁹ CFU/mL, the concentration of SS1 is 9.8×10⁷ CFU/mL, the concentration of the mixed LC1 and SS1 (LC1/SS1) is 8.0×10⁸ CFU/mL, and the concentration of the mixed PC2 and SS1 (PC2/SS1) is 7.6×10⁸ CFU/mL.

### Example 2 Analysis of SOD-Like Activity

The following experimental method is described in Marklund et al., 1974, and the SOD-like activity was quantified by the inhibition of pyrogallol oxidation. Pyrogallol is very stable below pH 7, but pyrogallol will undergo self oxidation, produce superoxide anion, and produce orange purpurogallin if pH value is above 7. Meanwhile, the superoxide anion will be disproportionated if the SOD-like activity is present, and the self oxidation rate of pyrogallol will be inhibited, so that the SOD-like activity can be calculated. One unit (U) of SOD-like activity refers to that the amount of enzyme can inhibit 50% of the self oxidation rate of pyrogallol per minute, and UV-Vis spectrophotometer detects the change of absorbance every 10 seconds in 1 minute at 420 nanometers (nm). Control group is 0.5% (w/v) miracle fruit matrix prepared from Preparation 2, experimental group is the same as Example 1 that 10¹⁰ CFU LC1, 10¹⁰ CFU PC2, 10¹⁰ CFU PC3, 10¹⁰ CFU PC4, 10⁹ CFU LA1, 10⁹ CFU SS1, 6×10⁹ CFU LC1/4×10⁹ CFU SS1, and 6×10⁹ CFU PC2/4×10⁹ CFU SS1 were respectively cultured in 100 mL of 0.5% (w/v) miracle fruit matrix obtained from Preparation 2 at 37° C. for 48 hours.

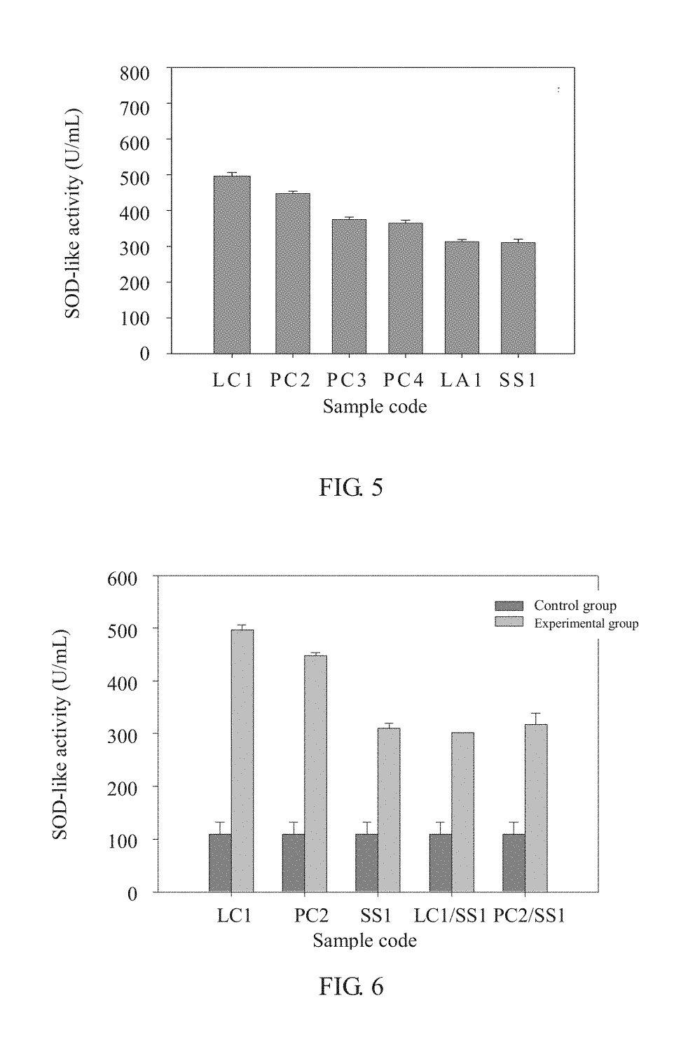

As shown in FIG. 5, LC1 has the highest SOD-like activity 496.64 U/mL, followed by PC2 447.49 U/mL, PC3 375.07 U/mL, PC4 364.72 U/mL, LA1 312.99 U/mL, and SS1 310.40 U/mL. As shown in FIG. 6, control group, 0.5% (w/v) miracle fruit matrix, has the SOD-like activity 109.8 U/mL; experimental groups after fermentation: LC1 has the highest SOD-like activity 496.64 U/mL, followed by PC2 447.49 U/mL; SS1 310.40 U/mL, LC1/SS1 310.30 U/mL, and PC2/SS1 316.88 U/mL has lower SOD-like activity compared to LC1 and PC2. Therefore, the SOD-like activity after culture and fermentation with lactic acid bacteria and the miracle fruit matrix has the synergistic effect.

### Example 3 Effects of SOD-Like Activity on Miracle Fruit Matrices of Different Concentrations with Lactobacillus casei LC1

The best SOD-like activity of fermentation strain Lactobacillus casei LC1 was cultured with miracle fruit matrices of different concentrations prepared from Preparation 2 for investigating the effect of the SOD-like activity on the miracle fruit matrices of different concentrations. Experiment was divided into control group before culture and experimental group after culture. 10¹⁰ CFU of LC1 prepared from Preparation 1 was cultured with 100 mL 6.5% milk powder as 0% miracle fruit matrix, 100 mL of 0.5%, 2% and 5% (w/v) miracle fruit matrices prepared from Preparation 2 were respectively cultured with 10¹⁰ CFU of LC1 at 37° C. for 48 hours.

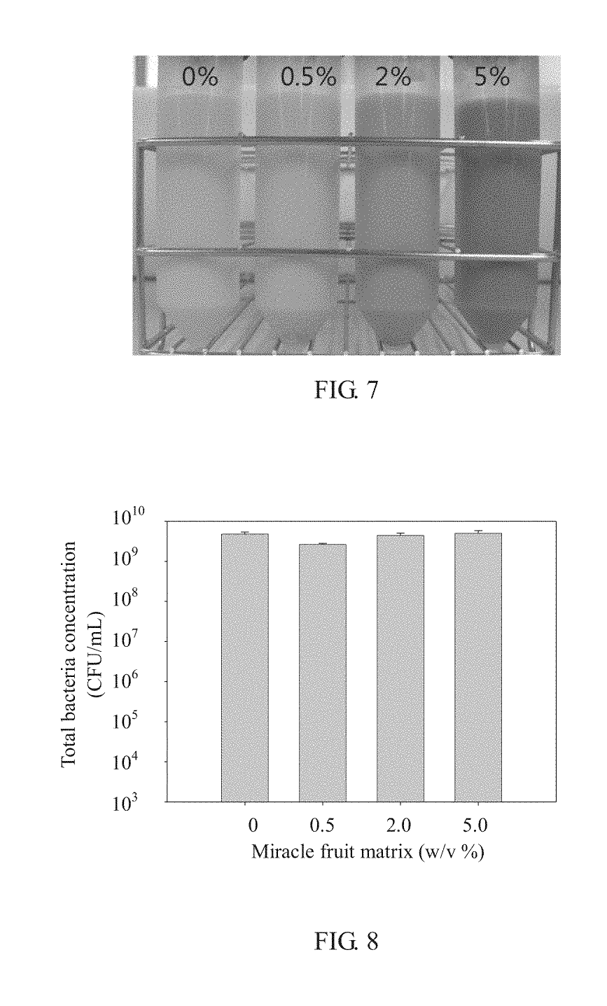

As shown in FIG. 7, after culture and fermentation LC1 of the miracle fruit matrices of different concentrations, the appearance of the miracle fruit matrices of different concentrations after fermentation were different. The higher the concentration of the miracle fruit matrix, the darker the miracle fruit matrix, the higher the viscosity of the miracle fruit matrix, and the precipitation is also more obvious compared to 0.5% (w/v) of the miracle fruit matrix of the control group. As shown in FIG. 8, the miracle fruit of different concentrations matrix have no effect on the growth of Lactobacillus casei LC1, and the total bacteria concentration of each group is about 10⁹ CFU/mL.

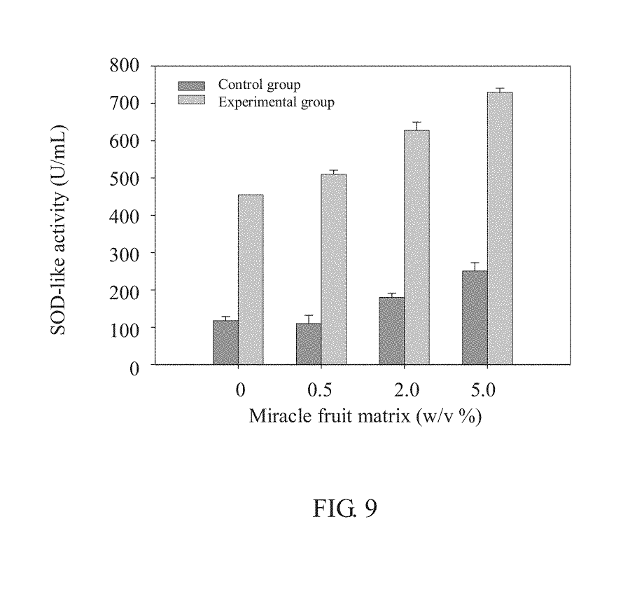

As shown in FIG. 9, the SOD-like activity is increased in each experimental group together with the increase of the concentration of the miracle fruit matrix. Four control groups are 0%, 0.5%, 2.0%, and 5.0% (w/v) miracle fruit matrices alone, and the SOD-like activities are 117.64 U/mL, 109.80 U/mL, 180.39 U/mL, and 250.98 U/mL, respectively. Four experimental groups are 0%, 0.5%, 2% and 5% (w/v) miracle fruit matrices respectively cultured with LC1, and the SOD-like activities of the four experimental groups are 454.9 U/mL, 509.8 U/mL, 627.5 U/mL, and 729.4 U/mL, respectively. The activities of the experimental group are 2.9, 3.6, 2.5, and 1.9 folds respectively compared to the control groups. As a result, 0.5% (w/v) miracle fruit matrix cultured with lactic acid bacteria has the highest SOD-like activity and has the synergistic effect.

### Example 4 Formulation of Synbiotic Tablet

The strains can be selected from, but are not limited to, ten strains from Table 1, single strain or multiple strains cultured with the miracle fruit matrix. In this example, a synbiotic comprising miracle fruit and probiotics was obtained from culturing the miracle fruit matrix and Lactobacillus casei LC1, and then the synbiotic was freeze-dried and ground into synbiotic powder. The final product of the tablets had 10⁸ CFU/g LC1, and each tablet contained 1 gram so that the tablet can be easily adjusted by the synbiotic powder. The synbiotic powder contained 10¹¹ CFU/g LC1 thereby accounting for 0.1% of the tablet. However, considering the loss of Lactobacillus casei LC1 during storage of the product and tableting, 1% of the synbiotic powder was added into the formulation. A first fruit powder is miracle fruit powder or miracle fruit-lemon juice powder; the miracle fruit and lemon juice were divided into groups at weight ratios of 1:1.5, 1:1, and 1:0 and were respectively freeze-dried and ground into miracle fruit-lemon juice powder. The tablet needs 30% of the miracle fruit having the taste-modifying ability which is the basic formulation. The formulation of the tablet is shown in Table 2.

TABLE 2
Tablet formulation of different miracle fruit-lemon
juice powders

| | miracle fruit:lemon juice | | | |
|---|---|---|---|---|
| | 1:1.5 | 1:1 | 1.5:1 | 1:0 |
| Different ratios of | 75 | 60 | 50 | 30 |
| miracle fruit-lemon juice | | | | |
| powders (w/w, %) | | | | |
| Synbiotic powder (w/w, %) | 1 | 1 | 1 | 1 |
| Corn starch (w/w, %) | 24 | 39 | 49 | 69 |
| Total weight (w/w, %) | 100 | 100 | 100 | 100 |
| Texture | very sour | sour | slightly | slightly |
| | | | sour | astringent |

The tablets can be made at four different ratios of miracle fruit-lemon juice powder (total weight 0.3 grams), and the higher ratio of lemon juice, the sourer the texture. Ratios 1:1 and 1.5:1 are more acceptable among the three ratios of miracle fruit-lemon juice powder, but since the tablet (0.3 grams) under test is one third of the final tablet product in weight and the total surface area is smaller than the final tablet product, the 1:1 ratio might be unacceptable to consumers in taste. Therefore, the ratios of miracle fruit-lemon juice powder 1.5:1 and 1:0 miracle fruit powder alone were chosen for the following formulation.

### Example 5 Formulation of Multiple Fruit Tablets

To add sweet flavor, and to achieve natural, low-calorie, sugar-free/low-sugar health supplements, second fruit powders produced from drying and grinding fruits including banana, tomato, pineapple, and/or pitaya were added into the tablet, and the textures of tablets were estimated.

(1) The Tablet Formulation of the Miracle Fruit-Lemon Juice Powder and the Second Fruit Powder

The tablet formulation of the miracle fruit-lemon juice powder and the second fruit powder was to reduce the corn starch of excipient (reduced from 50% to 25% in tablet 2). The tablet needed 30% of the miracle fruit and 10⁸ CFU/g of Lactobacillus casei LC1 of the synbiotic powder, the amount of the miracle fruit and LC1 was constant, so content of the second fruit powder was adjusted to 24% of the tablet.

TABLE 3
The tablet formulation of the miracle fruit-lemon juice powder
and the second fruit powder

| | | | | |
|---|---|---|---|---|
| Miracle fruit-lemon | 50 | 50 | 50 | 50 |
| juice powder (1.5:1) | | | | |
| (w/w, %) | | | | |
| Synbiotic powder | 1 | 1 | 1 | 1 |
| (w/w, %) | | | | |
| Second fruit powder | 24 | 24 | 24 | 24 |
| (w/w, %) | (tomato) | (pineapple) | (banana) | (pitaya) |
| Corn starch (w/w, %) | 25 | 25 | 25 | 25 |
| Total weight (w/w, %) | 100 | 100 | 100 | 100 |
| Texture | slightly | slightly | slightly | slightly |
| | sour | sour and | sour and | sour and |
| | | sweet | smooth | smooth |

As shown in Table 3, the second fruit powders including tomato powder, pineapple powder, banana powder or pitaya powder were added in to the tablet, and the above four formulations were tableted and had slightly sour lemon texture. The tablet containing pineapple has sweet flavor, and the tablets containing banana or pitaya have smooth texture.

(2) The Tablet Formulation of the Miracle Fruit Powder and the Second Fruit Powder

The tablet formulation of the miracle fruit powder and the second fruit powder was to reduce the corn starch of excipient (reduced from 50% to 25% in tablet 2). The tablet formulation containing the miracle fruit prepared from Preparation 2 and 10⁸ CFU/g of Lactobacillus casei LC1 of the synbiotic powder was constant, so the second fruit powder was adjusted to 44% of the tablet.

TABLE 4
The tablet formulation of the miracle fruit powder
and the second fruit powder

| | | | | |
|---|---|---|---|---|
| Miracle fruit powder | 30 | 30 | 30 | 30 |
| (w/w, %) | | | | |
| Synbiotic powder | 1 | 1 | 1 | 1 |
| (w/w, %) | | | | |
| Second fruit powder | 44 | 44 | 44 | 44 |
| (w/w, %) | (tomato) | (pineapple) | (banana) | (pitaya) |
| Corn starch (w/w, %) | 25 | 25 | 25 | 25 |
| Total weight (w/w, %) | 100 | 100 | 100 | 100 |
| Texture | tomato | pineapple | banana | smooth |
| | flavor | flavor, | flavor and | |
| | | slightly sour | smooth | |
| | | and sweet | | |

As shown in Table 4, the above four formulations can be tableted and each formulation has a different flavor depending on the different second fruit powders. The texture of the miracle fruit with pineapple is great, the texture of miracle fruit with tomato is not so appealing, both textures of the miracle fruit with banana or pitaya are smooth. As a result, pineapple and pitaya having the advantage of color were chosen for adjusting texture and color, and for the following formulations.

(3) The Tablet Formulation of the Miracle Fruit-Lemon Juice Powder and Multiple Second Fruit Powders

The second fruit powder accounted for 24% of the tablet, so the percentage of pineapple and pitaya was adjusted to under 24% of the tablet.

TABLE 5
The tablet formulation of the miracle fruit-lemon juice
powder and multiple second fruit powders

| | Group | | | |
|---|---|---|---|---|
| | Group 1 | Group 2 | Group 3 | Group 4 |
| Miracle fruit-lemon juice | 50 | 50 | 50 | 50 |
| powder | | | | |
| (1.5:1) (w/w, %) | | | | |
| Synbiotic powder (w/w, %) | 1 | 1 | 1 | 1 |
| Pineapple powder (w/w, %) | 14 | 16 | 19 | 21 |
| Pitaya powder (w/w, %) | 10 | 8 | 5 | 3 |
| Corn starch (w/w, %) | 25 | 25 | 25 | 25 |
| Total weight (w/w, %) | 100 | 100 | 100 | 100 |

The above four groups can be tableted, and the texture of the four groups are nearly the same. The larger the amount of pitaya, the darker red the tablet. The formulation of group 2 having the closest color of the miracle fruit is chosen for manufacturing test product.

(4) The Tablet Formulation of the Miracle Fruit Powder and Multiple Second Fruit Powders

The second fruit powder accounted for 44% of the tablet, so the percentage of pineapple and pitaya was adjusted to under 44% of the tablet.

TABLE 6
The tablet formulation of the miracle fruit powder
and multiple second fruit powders

| | Group | | |
|---|---|---|---|
| | Group 1 | Group 2 | Group 3 |
| Miracle fruit powder | 30 | 30 | 30 |
| (w/w, %) | | | |
| Synbiotic powder (w/w, %) | 1 | 1 | 1 |
| Pineapple powder (w/w, %) | 29 | 34 | 39 |
| Pitaya powder (w/w, %) | 15 | 10 | 5 |
| Corn starch (w/w, %) | 25 | 25 | 25 |
| Total weight (w/w, %) | 100 | 100 | 100 |

The above three groups can be tableted, and the textures of the four groups are nearly the same. The larger the amount of pitaya, the darker red the tablet. The formulation of group 2 having the closest color of the miracle fruit is chosen for manufacturing the test product.

(5) Taste-Modifying Test

The test products of Table 5 Group 2 and Table 6 Group 2 were tested by the method of Kurihara and Beidler published in Science, 1968. The tablet was kept in mouth until fully decomposed, and then 0.02 M citric acid kept in mouth could taste sweet. After rinsing the mouth by drinking water, 5% sucrose kept in mouth could not taste sweet, which means that the tablets of the test products have the taste-modifying ability as an equivalent of 2.5 grams sucrose.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for preparation of a synbiotic comprising miracle fruit and probiotics, the method comprising following steps:
(1) preparing miracle fruit and mixing the miracle fruit with water to obtain a miracle fruit solution;
(2) adjusting a pH value of the miracle fruit solution to range from 5 to 8;
(3) adding milk powder to the miracle fruit solution after adjusting the pH value to obtain a miracle fruit matrix; and
(4) culturing the miracle fruit matrix and the probiotics to obtain the synbiotic comprising the miracle fruit and the probiotics.

2. The method as claimed in claim 1, wherein the weight percent concentration of the miracle fruit in the miracle fruit solution is from 0.1% to 10% in the step (1).

3. The method as claimed in claim 2, wherein the weight percent concentration of the miracle fruit in the miracle fruit solution is from 0.5% to 5% in the step (1).

4. The method as claimed in claim 1, wherein the pH value of the miracle fruit solution is from 6 to 7 in the step (2).

5. The method as claimed in claim 1, wherein the weight percent concentration of the milk powder in the miracle fruit matrix is from 1% to 10% in the step (3).

6. The method as claimed in claim 1, wherein the time for culture is from 24 to 72 hours in the step (4).

7. The method as claimed in claim 1, wherein the method in the step (4) further comprises:
processing sterilization after culturing the miracle fruit matrix and the probiotics.

8. The method as claimed in claim 1, wherein the probiotics are selected from the group consisting of Lactobacillus casei, Lactobacillus paracasei subsp., Lactobacillus acidophilus, Streptococcus salivarius subsp. thermophilus, and any combination thereof.

9. A synbiotic comprising miracle fruit and probiotics prepared from a method comprising following steps:
(1) preparing miracle fruit and mixing the miracle fruit with water to obtain a miracle fruit solution;
(2) adjusting a pH value of the miracle fruit solution to range from 5 to 8;
(3) adding milk powder to the miracle fruit solution after adjusting the pH value to obtain a miracle fruit matrix; and
(4) culturing the miracle fruit matrix and the probiotics to obtain the synbiotic comprising the miracle fruit and the probiotics.

10. A tablet of synbiotic comprising miracle fruit and probiotics, the tablet comprising:
a synbiotic powder obtained by drying and grinding the synbiotic comprising miracle fruit and probiotics as claimed in claim 9;
a first fruit powder; and
an excipient.

11. The tablet as claimed in claim 10, wherein the weight percent of the synbiotic powder in total weight of the tablet is from 0.1% to 1% (w/w).

12. The tablet as claimed in claim 11, wherein the first fruit powder is miracle fruit powder or miracle fruit-lemon juice powder; wherein the miracle fruit-lemon juice powder is obtained from mixing miracle fruit and lemon juice at a weight ratio ranging from 1:1.5 to 1.5:1 after drying and grinding.

13. The tablet as claimed in claim 11, wherein the tablet further comprises a second fruit powder, and the second fruit powder is selected from the group consisting of tomato powder, pineapple powder, banana powder, pitaya powder, and any combination thereof.

14. The tablet as claimed in claim 13, wherein the tablet further comprises Bacillus, Bifidobacterium, Enterococcus, Lactobacillus, Saccharomyces, Sporolactobacillus, Streptococcus, Clostridium, Monascus, Propionibacterium, Candida, or any combination thereof.
